(19) United States
(12) Patent Application Publication
(10) Pub. No.: US 2005/0043608 A1
(43) Pub. Date: Feb. 24, 2005

(54) Method and apparatus for non-contact monitoring of cellular bioactivity

(76) Inventor: Yousri Mohammed Taher Haj-Yousef, Al Ain (AE)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 10/775,813

(22) Filed: Feb. 9, 2004

(30) Foreign Application Priority Data
Aug. 18, 2003 (AE) 265/2003

(51) Int. Cl. A61B 5/05

(52) U.S. Cl. 600/407

(57) ABSTRACT

A method and an apparatus for non-contact (non-invasive) monitoring the cellular bioactivity by means of tracking the impedance changes which occur due to the transition movements and reactions of ionized molecules across the cellular membrane, or by tracking the cellular movement or flow within the body. Tracking the impedance changes is based on the transmission line impedance match and mismatch phenomena, by means of monitoring the slight match and mismatch variations between the resultant impedance of the body being monitored and the output impedance of a stable source of EMW.

### BACKGROUND OF THE INVENTION

The present invention pertains to a method for non-contact (non-invasively) monitoring of cellular bioactivities by three means:
a)—Monitoring impedance variations which occur due to the transition movements of ionized molecules across the cells membrane.
b)—Monitoring impedance variations which occur due to differential concentrations of ionized molecules at both sides of the cells membranes; this is the basis of cellular impedance.
c)—Monitoring impedance variation which arise from cellular movements or flow within the body.

Monitoring micro-movement activities of concealed, non-metallic objects in free atmosphere, is extremely difficult with previously applied technologies.

Current motion detectors are classified in two general categories:

First is the active type—a technology based on transmitting a signal of ultrasound, laser, or electromagnetic waves (EMW) at the target of interest, and comparing the transmitted signal with signal reflected from the target to detect movements (eg. Radar).

Second is the passive type—Such as, infrared motion detector used in access control, is based on capturing target activities such as the heat changes.

The aforementioned techniques have limited sensitivity due to the need to transmit the detection signal through the ambient atmosphere, where ambient noise and interference may distort the signal and thereby compromise the accuracy of the detection system.

The creation of a closed system that can monitor the movements activity of concealed bodies and objects has been achieved by means of exploiting the transmission line impedance match and mismatch phenomena. The patent to Haj-Yousef (U.S. Pat. No. 6,359,597), has demonstrated the method that can be used to achieve a closed system motion detector. The prototype which was used in support of the said Haj-Yousef Patent reached a sensitivity of about 100 parts per million (PPM), therefore it was intended to monitor the relatively large object movements.

Sensitivity has been estimated by the ratio of the impedance variation (ΔZ), which occurs due to the target movements relative to the total impedance (Z) of the surrounding media, which contains that target.
Sensitivity (S)=ΔZ/Z

A subsequent prototype was completed which demonstrated a sensitivity of about 0.1 PPM. This means that for 50-ohm load impedance, a 5 micro-ohm impedance variation has been traced.

Laboratory observation with subsequent prototypes indicate that sensing micron and even the nano-scale movements of objects is possible. Work to date with this technology indicates the potential for achieving sensitivity in the range of a few parts per trillion (PPT).

A number of un-expected results have been observed in this work, these include (by way of example and not limitation), monitoring the movements of hidden isolators (Glass, Plastic, sponge . . . etc.) through a non-metallic or partially metallic barrier; a similar sensitivity is observed when monitoring movements of metallic objects. Because the isolators are negatively effecting the resultant impedance of the surrounding (inspected) media when the isolators occupies part of it, which therefore reduces the components that characterizes the resultant impedance of the said media.

Sensitivity is not affected by normal or artificial airflow even if the scrutinized media is the ambient air. While the air moves, the surrounding air immediately occupies the same place. Therefore the airflow doesn't produce any variations in the resultant impedance.

Additionally, sensitivity is not effected by the presence flame within the inspected area.

From the aforementioned observations, additional important applications have been identified.

### SUMMARY OF THE INVENTION

When stable high frequency (HF) electromagnetic waves (EMW) travel outwardly along a transmission line (coaxial cable, dual strip-lines . . . etc) to a balanced antenna which surrounds (adjacent) the scrutinized media that contains the target of interest, a specific power value of the EMW will be released from the transmission line and completely absorbed by the load (media being monitored) due to the impedance match level between the load and the EMW source. Any impedance mismatch results in a different specific power value which will not be released from the transmission line, rather, when the mismatch waves reaches the end of the transmission line, it is reflected back toward the EMW source.

It is well known that the maximum power absorbed by the load occurs when the load impedance and the EMW source impedance are equal (fully matched). Therefore monitoring these two power values provides an opportunity to obtain values relating to the actual degree of match/mismatch.

The first power value, which has been completely absorbed by the load due to the impedance match, is called the forward (incident) value and it can be sampled before being released from the transmission line. The second power value, which is reflected in phase and not released from the transmission line due to the impedance mismatch, is called the reflected value. These two bi-directional power values are generated only inside the transmission line, and they manifest on the basis of the transmission line impedance match and mismatch phenomena. These two power values can be sampled instantly and precisely by passing the transmitted EMW through a bi-directional coupler, which is connected in series within the said transmission line.

A matching network is used to buffer and tune the load impedance to approximately 50-Ohm (Ω), which is equal to the system impedance (transmission line and EMW source). The bi-directional coupler samples the instant power values of the forward and reflected waves in voltage form, such as forward voltage VF, and the reflected voltage VR. These two voltage values are totally free from any ambient electromagnetic interference (EMI) or noise, because the forward power value is sampled before being released from the transmission line, and the reflected power value is never released from the said line. Any slight movements within the inspected media by the target of interest will vary the resultant characteristic impedance of the said media, which will also vary positively or negatively the degree of impedance match and mismatch. By using two DC (direct current) blocking capacitors functioning as a high pass filter (HPF) connected to the rectified outputs of the bi-directional coupler, the variable components of the VF and VR voltages, which contain the useful indication about the target movements (activities), are only crossing these capacitors toward the next processing stage. The variable components of the VF and VR voltages have a symmetric non-proportional relationship, which means that when the VF signal increases, the VR signal decreases and vise versa, and a combined differential signal occurs. By directing these two extracted variable voltages to the inputs of the Differential amplifier (DA) or to what is so called “Instrumentation amplifier” (IA); an indication of the target movements and activities can be obtained and a close type monitoring system is established.

By way of example and not of limitation, the method of the present invention comprises the usage in the following applications:
1. Non-contact monitoring of bodily Hemodynamics:
   The Hemodynamics of the blood flow within the capillaries or main vessels (veins/artery) contains important data which reflects mechanical activities of the vital organs. Since the heart and the lung are mechanical organs, a way to detect their mechanical performance is vital and will become an essential diagnostic tool.

The principal present-day methods used to monitor vital hemodynamic activities within the body are:
a)—Heart rate sensors (Photo-Plethysmography—PPG): this is used to measure the cardiovascular pulse wave that's found throughout the human body. The pulse wave results in a change in the volume of arterial blood with each pulse beat. This change in blood volume can be detected in peripheral body parts such as fingertips or ear lobes. The technique consists of an infrared Light Emitting Diode (LED), which illuminates the tissue and Light Sensitive Detector (LSD), which has been tuned to the same color wavelength as the LED, and therefore it detects the amount of light absorbed by the tissue. The beats per minute are calculated by timing the width of a pulse and scaling up to a rate of beats per minute.
b)—Pulse Oximetry: The principle of pulse Oximetry is based on the red and infrared light absorption characteristics of the oxygenated and the deoxygenated hemoglobin. Oxygenated hemoglobin absorbs more infrared light and allows more red light to pass through. This method uses two LED's, red and infrared instead of one infrared LED as in PPG, in a similar way it monitors the light absorption of blood within the fingertip or the ear lobe to acquire an indication of respiration activity.
c)—Ultrasonic blood flow Doppler: a transducer probe is used to beam ultrasonic waves into a specific vessel. The beam is reflected with slight frequency changes that are due to the speed variations of the blood flow; by tracking these frequency variations, the speed (non-directly the volume) of blood flow can be monitored.

Optical methods used for blood flow measurements have many limitations and disadvantages; for example, to obtain a suitable reading, in most cases it is necessary to warm the hands by rubbing to increase the blood flow.

The following physical factors and diseases also disturb optical readings: Malpositioned Sensor (Penumbra Effect), Light Interference, Sensor site temperature, Fingernail Polish, skin color, Motion, Burns, Venous Motion, Venous Pulsations, Venous Congestion, Sickle Cell Anemia, Pressure Necrosis, Fetal Hemoglobin, Intravascular Dyes, Bilirubin, Low Perfusion, Localized Hypoxemia, Carboxyhemoglobin, Methemoglobin, Low oxygen saturation (SaO₂ less than 70%), Magnetic Resonance Imaging (MRI), Electrocautery.

Optical method used to detect the absorption variations of specific color, cannot detect both veinal and arterial blood at the same symmetrical sensitivity, as the color of the oxygenated and deoxygenated blood is highly variable from person to person.

The following are the disadvantages and the limitations of the Doppler blood flow technique:

The Doppler transducer should be well secured to the vessel to be inspected; this disturbs normal blood flow characteristics. Moreover portable Doppler equipment can be only used to monitor the visible, superficial vessels. Doppler is still not able to monitor capillary blood flow such as fingertip or the ear lobe. Similar to optical method limitations, Doppler cannot recognize blood flow variations of less than 1% over the full-scale bandwidth. The big physical size of the Doppler transducer is an additional disadvantage, specially for long-term monitoring.

At present, the traditional electro-cardio graph (ECG) outlines only five curves/three peaks (QRS, T and P). Diagnosing the ECG abnormality can be obtained by monitoring changes in the observed signals.

The ECG is now used to identify a limited number of heart problems such as arrhythmia. The possibility of spurious results exist as in cases where the ECG show normal heart activity while the heart is completely dead, as in the case of electromechanical cardiac dissociation.

The present invention has overcome limitations associated with previous monitoring techniques. It is not influenced by any kind of ambient EMI or noise. Blood flow variations in the range of few parts per billion (PPB) can be monitored. Additionally, diseases which affect blood contents or intensity, do not bias the results.

The subject invention can provide non-invasive monitoring by fastening a small insulated probe at any point on the body and may even be placed above some layers of clothing or shoe.

This technology can be used to monitor blood flow waveform within any vein or artery to determine clotted vessels or arteriosclerosis. Positioning the sensor over an area of skin that contains the capillaries allows provision of extensive data concerning heart/Hemodynamic activities. The capillaries are the locus where the veins and the artery are coupled. Therefore this area contains signals which can be monitored to obtain information regarding heart and circulatory system function.

The sensitivity of the current prototype has reached a level that can trace the activities for any of the heart components such as the valves or the cavities contractions within the same magnified graph. At the same time, heartbeat rate and respiration activity can also be precisely monitored, since the inspiration causes thoracic pressure to decrease inside vessels; this slight pressure decrease is not available by other non-contact techniques.

Over few millimeters of skin at rest, a blood flow variations as low as few nano-liter can be traced by the subject technology.

The subject invention will provided a robust, reliable, and easily applied analytical tool in the field of medical diagnosis.
2. Non-contact monitoring nervous system bioactivities:

This invention (basic sensing technology) may be beneficially applied to a variety of applications in a broad spectrum of industries.

Before explaining the value of this conclusion, it is important to explain how this been deduced.

While testing the slight voluntary movements of the finger, by fully relaxing the palm above a 5 centimeter thick granite tile, and the motion sensor was placed beneath the said tile. It has been observed that the system is distinguishing the initial movement action before it is perceived by the subject. Apparently, the finger starts the movement. This infers that the sensory/perceptual threshold is higher than the muscle threshold; this indicates that muscle movement occurs without conscious perception of the brain, rather activity precipitating movement, appears in the peripheral nervous system. When the sensor is positioned posterior region of the neck, a few millimeters from the Medulla Oblongata at the top of the Spinal Cord, the system has monitored many signals synchronized with neural/sensory or motor activities such as the slight movements of the toe. Such observation indicate that the system is capable to monitoring more than simple muscle movement.

When head is positioned between two insulated sensor electrodes as depicted in FIG. 4, and the electrodes are distanced from the scalp by one-centimeter thick sponge, the sensors report brain signals which appear to reflect instant brain response to visual stimuli presented to the subject. The resulting graph appears similar to the standard signals obtained by classical EEG; eg. delta waves.

The subject sensing system has demonstrated, under different circumstances, that it is unaffected by any electric or magnetic interference or noise.

Additionally, the detected brain signal has been instantly synchronized with the observed brain activity sensed by conventional methods, thereby eliminating the possibility that the subject sensing technique is monitoring only blood flow variations as a result of the brain activity, functional MRI has shown that the pattern excitation results in maximum blood flow in the brain with a time delay of approximately 6 seconds.

From the preceding examples and observations this system has demonstrated the capability of sensing neural communications by monitoring molecular activity which occurs due to the chemical reactions within the neurons.

In the mechanics of the central nervous system(CNS), the communications between the brain and sensory or the motor neurons, is achieved by creating a chemical reactions within the neuron membrane where ionized potassium, sodium and chloride molecules are moving on both sides of the neuron membrane; these molecules create differential concentrations of polarized ions inside and outside the neuron which result in the synapse firing. This molecular movement is repeated many times along the nerve fibers until the massage reaches its target.

A collection of living cells always has properties of resistance, displacement capacitance, and impedance. When the cell is stable or at rest, there is a 70 mV potential between inside and outside of the cell, potassium ions are concentrated inside the cell and sodium and chloride ions are concentrated outside the cell. The cell at rest also has an electric resistance of about 10 kΩ/cm, and it has about 1 kΩ/cm at action. Therefore the cell bioactivity produces three effects: molecular movement, impedance variation, and electrical potential.

Impedance Cardiography (Impedance Plethysmography), also known as thoracic electrical bio-impedance, is a method that has been used to measure superficial impedance changes in order to monitor internal bioactivity. Impedance measurement is achieved by introducing an electric current into the body surface and then measuring the corresponding voltage. The ratio of voltage to current gives impedance (ohms law). Any change in the region's conductivity produces a change in the resultant impedance, which is proportional to the amount of current flowing in that region. Separate electrode pairs for introduction of current and measurement of voltage are used; the outer electrode pair is used to introduce the current, the voltage is measured across the inner electrode pair. This method employs direct electrical contact with the patient. At present the sensitivity is limited to about 0.1 to 0.01 ohm, therefore this method has been applied in monitoring large physiological activities.

A key feature of the present invention is the elimination of direct electrical contact with the patient; the subject sensing technology invention, facilitates acquisition and interpretation of micro and nano-ohm variations in load impedance via a closed monitoring system.

In view of the previously reviewed technical functionality, potential applications of the subject sensing technology invention, would not be limited by, but would include the following:
a)—Non-invasive encephalograph: which monitors brain bio-activities by tracking impedance variations and following molecular movement inside the brain. Monitoring brain bio-activities can be used in non-medical sectors also, such as criminal investigations; by so called “Brain fingerprinting”, a method created by Dr. Lawrence Farwell to identify the perpetrator of a crime, by associating physical evidence from the crime scene with the evidence stored in the brain, and measuring brain wave responses to crime-relevant words or pictures presented to the suspect.
b)—Non-invasively monitoring nervous system bio-activity: this can help in the diagnosis of nervous system or muscle diseases. This subject sensing technology may be of significant value in realizing intelligent prosthetic limbs and sensory organs by acting as the interface for direct neural activation of prosthetic devices.

Functional magnetic resonance imaging (f-MRI) is also a method used to study the blood-flow volume inside the brain and thereby, indirectly, brain activity. By taking many short interval anatomic pictures of the brain, blood-flow to different regions can be observed as changes in the sizes of blood vessels. The assumption made here is that the areas of the brain, which are in use, will require more blood, and if they are using more blood the blood vessels will be larger. Researchers look at changing sizes of the blood vessels then infer that particular regions of the brain are being used at particular times.

Many methods are currently used to monitor the brain bio-activity, such as the Electro-encephalograph (EEG), which employs a dozens of bulky electrodes being attached to the scalp through salted gluing gel; this technique is used for capturing the brain bioelectricity. Due to the ambient EMI and noise, the EEG monitors only brain signals that are larger than 1 micro-volt can be distinguished.

Most of the current methods used to monitor cellular bio-activity are focused on direct capture of the cellular bio-electricity; as an example, Electro-cardiography (ECG), Electro-encephalography (EEG), Electro-myography (EMG), Electro-nervography (ENG), Electro-gastrography (EGG). etc.

Tracking electrical signals which occur in a specific combination of cells (tissue) has many limitations which degrade the value of the extracted data. For example, different sources of bio-electricity are crossing the same frequency bandwidths, as in the EMG and ENG. Narrowing the monitored bandwidth is one possible solution, but it omits much relevant data. Also, the level of sensitivity is impacted by the level of the ambient EMI and noise, which is involved in achieving good signal to noise ratio (SNR).

Additionally, to monitor cellular bio-electricity, a direct electrical contact with the patient is currently required. Therefore, additional precautions for the patient safety are vital to avoid any threat to the patient from an accidental system breakdown which could result in an electrical shock as current monitoring systems are usually powered from the main AC power line. Attaching contact electrodes to the patient skin results in poor stability and introduces extraneous noise. The following are limitation factors: skin preparation (hair shaving), air bubbles within the conducting gel, electrode and lead motion artifacts, current leakage, electrode polarization specially in long term monitoring, large skin to electrode impedance, perspiration, EMI, Electrocautery, MRI . . . etc.

The presented invention provides a tool for non-contact monitoring of bio-activities of the central or peripheral nervous system (brain, spinal cord and spinal nerves. etc), the very low sensing threshold can track even very low power brain wave activity (brain whispers).

This method overcomes many limitations of currently deployed technologies by monitoring directly and non-reactively extremely low biological effects of microscopic particles within living cells.
3. Fetal Cardiography:

Potential applications include fetal-cardiography monitoring the fetal heart rate and the maternal contractions by tracking vital bioactivities of fetal organs.
4. Insect cardio, respiration, and general activity graph:

All types of insects conduct respiration and have blood circulation. Monitoring such microscopic movements is of significant research interest, but extremely difficult to achieve. The presented invention has the capability of monitoring this activity while the insect is allowed free movement inside a ventilated chamber under no stress of direct sensor contact.

This can be achieved by placing the anesthetized insect above the insulated sensing plate for establishing the insects reference baseline, subsequently a spectrum analyzer is used to evaluate the normal Insect bioactivity by determining its frequency bandwidths. Then, while the insect in its normal activities [applying Fourier theorem using band-pass filters (BPF) via digital signal processing (DSP)] the target frequencies bandwidth, are tracked and extracted from the signal complex which contains all of the insect signal artifacts including the bioactivities.

Monitoring the insect's vital signs within any recommended ambient condition is vital and will expand our knowledge of insect biology. The subject technology will provide an essential development tool in the field of pesticides technology.

### BRIEF DESCRIPTION OF THE DRAWINGS

FIG. 1 is a block diagram of the preferred embodiment

FIG. 2 illustrates an embodiment of the invention for monitoring the superficial hemodynamics through the finger; likewise monitoring can be obtained through any other region of the body surface.

FIG. 3 illustrates a cross-sectional view of the finger being monitored. The highest EMW intensity is found inside the finger and near the surrounding electrodes. The sensitivity grading lines (regions) also illustrated.

FIG. 4 illustrates an embodiment of the invention for monitoring the brain bioactivities (a single channel is shown); the probe assembly looks like the normal headphone.

FIG. 5 illustrates an embodiment of the invention for monitoring the bioactivities of the central and peripheral neural system (spinal cord and spinal nerves . . . etc).

FIG. 6 illustrates an embodiment of the invention for monitoring the vital signs of the fetal organs, such as the heart, the lung, and the maternal contractions.

FIG. 7 illustrates the preferred shape of the overlapped transmitting antenna that can be used to monitor the insect's bioactivity. The electrodes can be constructed from a copper clad fiberglass board (printed circuit board), wherein the 1.6 mm fiberglass can perform the required insulation.

FIG. 8 illustrates the assembly details of the preferred adjacent-type transmitting antenna 48 (sensor/probe).

FIG. 9 illustrates the internal details of the preferred ultra-narrow band pass filter, which comprises many parallel crystal ladder filter modules 34.

FIG. 10 illustrates an embodiment of the invention as a multi-channel monitoring of bioactivities.

FIG. 11 illustrates the preferred embodiment of the invention that maintains the high sensitivity while utilizing a low transmitting power of EMW.

FIG. 12 illustrates the preferred embodiment of the invention by adding non-directional coupler within the transmission line to produce a negative reference that reflects the instability of the produced HF EMW.

FIG. 13 illustrates the non-proportional characteristic of VF and VR over the impedance bandwidth of a tuned load. The sensitivity becomes maximum where the curves turn into exponentially sharp (VSWR=1 to 1.5).

FIG. 14 illustrates samples of the output graphs being obtained by the present invention, with the exception of graph (a). The remaining graphs are genuine, absolutely raw, and have not been processed by any means; the graphs were obtained in sequence using a single channel analogue to digital converter (ADC), then they were manually added to the said figure; the illustrated graphs were obtained by placing the sensing probe at various points of the body of a 39 years old man. The graphs were extracted using different frequency bandwidths, but more clinical research is still needed to define the best frequency bandwidths:
a) Represent standard ECG chart, and it is just shown here for the ease of comparison with the obtained graphs. Normally the raw ECG signal contains EMI, noises, and artifacts in addition to the useful signal. Therefore many signal processing is required to achieve an acceptable graph like the one shown here.
b) Represent the hemodynamic cardiograph obtained from the wrist, above the capillary vessels and through clothes, the equivalent QRS, T, and P peaks are obvious, they vary slightly from the ECG by the time bandwidth, since the ECG outlines heart's depolarization instead of monitoring heart's mechanical activity as in the presented invention. The corresponding bandwidth applied is from 1 to 25 Hz.
c) Represent the hemodynamic cardiograph obtained from the wrist, above the main vessels (artery and vein), a very tiny rhythmic curves are clear at the top of the systolic peak. The corresponding bandwidth applied is from 1 to 25 Hz.
d) Represent the hemodynamic cardiograph obtained from the thigh, a very tiny rhythmic curves are clear on the right side of the systolic peak. The corresponding bandwidth applied is from 1 to 25 Hz.
e) Represent the obtained heart beat cycle that can be used to calculate the heart beat rate, this is covered by the frequency bandwidth of 1 to 1.5 Hz.
f) Represent the obtained respiration cycle that covers the frequency bandwidth of 0.2 to 0.3 Hz. similar hemodynamic cardio and respiration graphs were obtained by putting a larger sensor (size of few centimeters) beneath a chair, through 10 to 15 cm thick non-metallic barrier between the body of the person sitting on the chair and the sensor probe. Such way of remote hemodynamic monitoring can be very useful in few critical cases were it is vital to react immediately; for instance monitoring aged passengers in airplanes, or drivers of critical transportations such as pilots, astronauts, train drivers . . . etc. As well as monitoring patients in Intensive Care Units (ICU).

The numbers in the drawing are:
1 is a HF oscillator;
2 is a HF power amplifier;
3 is an ultra-narrow band pass filter;
4 is a transmission line;
5 is a rectifying diodes;
6 is a bi-directional coupler;
7 is a matching network;
8 is a transmitting cable;
9 is a Balun (balanced to unbalanced transformer);
10 is a transmitting electrodes (sensor probe);
11 is an inspected region;
12 and 13 are forward VF and reflected VR voltages, respectively;
14 and 15 are RF suppression chokes;
16 and 17 are DC blocking capacitors—HPF;
18 and 19 are the load resistors of the HPF;
20 and 21 are the extracted wavering (variable) signals of the VF and VR voltages;
22 is a differential or instrumentation amplifier;
23 is a differential signal;
24 is an analogue divider;
25 are the divided outputs;
26 are active filters;
27 are output amplifiers;
28 are output ports;
29 is an electrical insulator;
30 are fixing arms;
31 are connecting wires;
32 are overlapped transmitting electrodes
33 is a multi-port HF power splitter;
34 is a crystal ladder filter module;
35 is a multi-port HF power combiner;
36 is a bi-directional coupler with HF outputs;
37 is an input filter (ceramic filter);
38 is a HF selective amplifier;
39 is a crystal ladder output filter;
40 is a HF demodulator;
41 is a HPF;
42 is a HF forward power;
43 is a HF reflected power;
44 is a non-directional coupler;
45 is a signal produced by the non-directional coupler;
46 is a linear amplifier;
47 is a negative reference;
48 is a transmitting antenna (transducer/probe)

### DETAILED DESCRIPTION OF THE PREFERRED EMBODIMENT

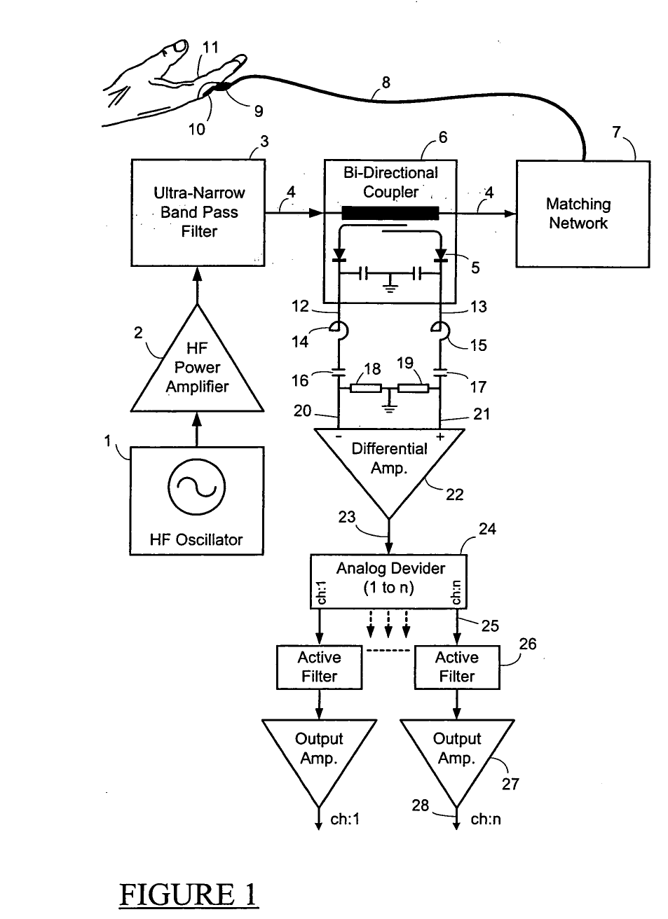

According to the present invention, the device can be described by referring to the drawings and more particularly to FIG. 1. The HF oscillator 1 is used to produce a fixed sinusoidal frequency, means to achieve a very stable and low noise EMW energy in the frequency range of 1 to 300 mega-hertz (MHz) with an output power of less than one milli-watt (mW).

The produced EMW is then amplified to the desired power level ranging from 1 to 100 mW by the HF power amplifier 2. The ultra narrow band pass filter 3 is used to clean the produced EMW from noises.

The purified EMW then passes through a bi-directional coupler 6, which is connected in series within a transmission line 4 (coaxial cable, dual parallel wires, or strip-lines). The bi-directional coupler 6 is used for instant sampling both the internals forward and reflected power values, which are generated inside the said transmission line 4. The EMW then passes toward the matching network 7, which is used to tune and buffer the 50-ohm (Q) impedance of the HF oscillator 1 with the characteristic impedance of the load 11 (the region of the body being monitored). Due to its structure, the matching network 7 furthermore will act as a harmonic reject filter, which can be built from any of the popular types L, PI, or T filter networks.

The released EMW from the matching network is then introduced to the said load directly by a balanced type-transmitting antenna 48, or it can be introduced to the load indirectly via transmitting coaxial cable 8 through the same antenna 48. The said antenna 48 as depicted in FIG. 8 consisted of pair of electrodes 10 made from insulated pieces of metallic sheets or wires. The balanced antenna 48 is connected to the coaxial cable 8 by balun 9 (balanced to unbalanced transformer).

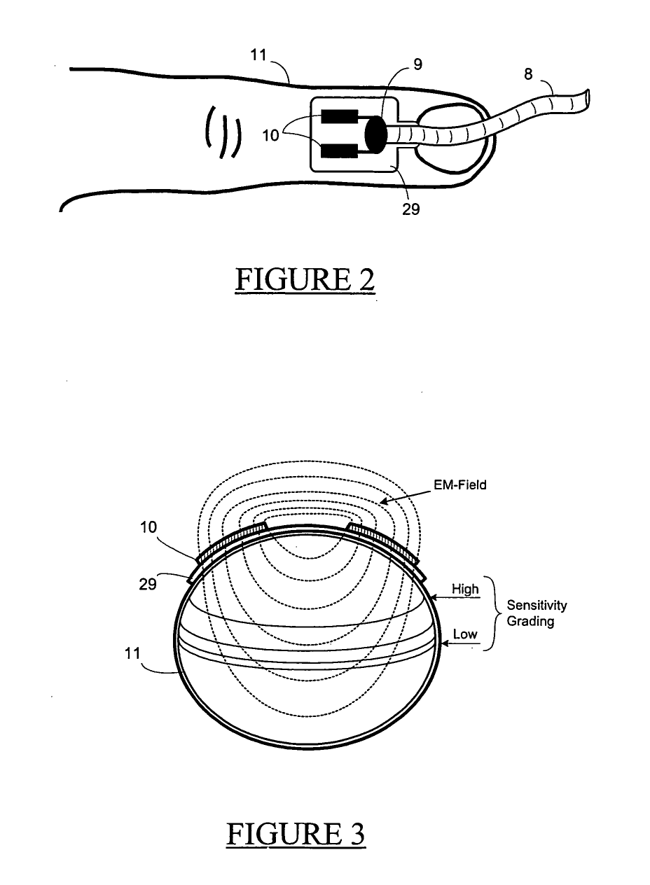

The geometry size of the electrodes 10 defines the preferred coverage area plus the desired sensitivity depth, wherein the larger electrodes (L and W) will cover more surface area, and the increase of distance (D) between both electrodes will increase the effective depth of the sensitivity within the media being monitored. For example: monitoring the superficial capillary blood flow within the finger as depicted in FIG. 2, an electrode length (L) of about 5 millimeters (mm) by a width (W) of about 3 mm, having a distance (D) between both of the electrodes of about 2 to 3 mm, has seemed to be sufficient to obtain a satisfying results.

A dual HPF consisting of capacitor 16, 17 and resistor 18, 19 connected to both outputs 12, 13 of the bi-directional coupler 6, the capacitors 16, 17 will only allow the variable (wavering) voltages to pass through, and the direct current (DC) will be rejected. The extracted variable voltages 20, 21 are imitating the impedance match and mismatch variations occurred in-between the load 11 and the HF oscillator 1.

The extracted variable voltages 20, 21 are then combined together by a Differential amplifier (DA) 22 or to what is so called instrumentation amplifier (IA). Any ambient EMI could even reach the transmitted antenna 48, affects both of the DA inputs 20, 21 evenly and by the same phase, therefore such external common noise will be highly rejected duo to the high common mode rejection ratio (CMRR) available for this type amplifier, which now has been reached to more than 134 decibels (dB). Therefore the resulted combined signal has become to be so pure and unsusceptible to any external EMI or noise.

The output signal 23 thereafter directed to an analog divider 24, which produces multi outputs 25 that each mirrors the same characteristics and parameters of the input signal 23. The analogue divider 24 in particular is required when the same signal contains many vital parameters and indications. For example the signal obtained by monitoring the hemodynamic activities of capillary vessels contain a lot of information about the mechanical heart activity beside the respiration cycle, therefore dividing the signal to two channels, each of them represents a specific activity that can be discriminated by limiting the expected frequency bandwidth. This is can be achieved by the subsequent use of an active filter 26. The active filter 26 can be established by means of operational amplifiers with a few passive components such as resistors and capacitors, or by using of modern computerized technology, such as the digital signal processing, these circuits can achieve the low pass, high pass, band pass, or band reject filter.

However the produced signal still needs to be amplified to a sufficient level that can drive the next analytical circuits, this can be performed by the output amplifier 27.

The general description explained above describes only the general functioning of the device. Utilizing standard readymade blocks, which are popular and widely available in the market cannot achieve sensitivity better than few parts per thousand only.

Consequently the system designer should take a few significant measures into consideration, which is so vital to achieve a system with an ultra high sensitivity.

The HF section (1-10) has become to be the most critical part that can define the final sensitivity. The design of HF oscillator requires more concernment about the noise floor. Manufacturers of the HF oscillators are now showing more attention for reducing the phase noise and for the enhancement of the long-term stability. Nevertheless as an example, an ultra-low-noise RF oscillator with a noise floor of about −174 dB has been established by the American Wenzel Inc. (ultra-blue low-noise oscillator series). Such oscillator is an excellent choice and exceeds the requirements, but it's output power of about 0.5 mw still very low.

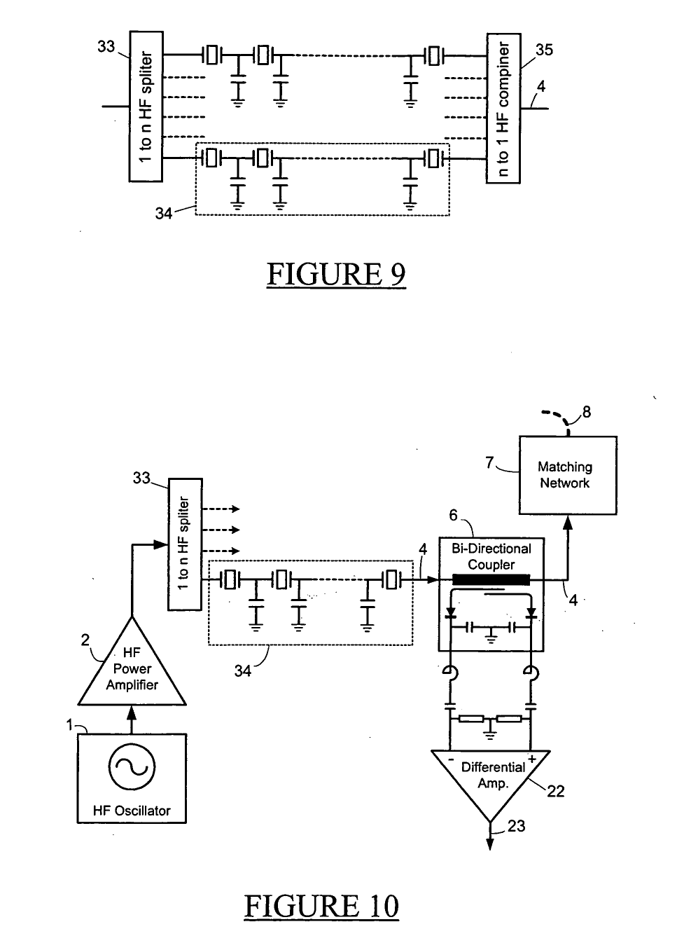

The produced EMW power will need amplifying to become usable. This can be achieved by using of HF power amplifier. Using any of the popular hybrid wide band RF amplifier for this stage is useless. Such type of amplifier produces a lot of noises, which contaminate the amplified EMW. The use of narrow-band (selective), and very low-noise HF amplifier 2 is vital to establish a high quality EMW. Nevertheless the produced EMW still need to be purified from the noises, which occurred internally by the amplifier and the oscillator circuits. Using of narrow band pass filter 3 will help, but the traditional LC (inductive and capacitive) resonant type will not achieve a satisfying quality, due to the limitation of there low quality factor, which cannot exceed a few hundreds. A quality factor in millions can only be established by using of the crystal ladder filters (CLF) 34 as depicted in FIG. 9. In general the present days crystals cannot tolerate driving powers above 10 mW.

In many applications it is required to use a higher power than the crystal limit. Splitting the EMW energy by a HF power splitter 33 to a few matched and isolated ports will allow dividing the high EMW power to many matched and paralleled multi-order CLF 34, thus by combining the filters outputs together by a HF power combiner 35, a very clean and noise-free high power of EMW is produced.

Few many relationships should be maintained by the system designer, which is needed to achieve the highest sensitivity. For instance the produced DC voltage value of VF 12 should be as large as possible, but to achieve a good differential symmetry between both voltage values of VF12 and VR 13, the coupling coefficient of the bi-directional coupler 6 should be in the range of 30 to 40 dB.

The sensitivity threshold of the DA (IA) 22 is limited to a few nano-volts (nV) due to the DA/IA self noise. One of the best IA that has 1.6 nV/₍ᵣₒₒₜ₎Hz self noise voltage which equals about 10 nV RMS (Root Mean Square) and for the bandwidth of 0.1 to 100 Hz is INA 166 made by Texas Instruments Inc. Therefore the lowest input voltage which is required to achieve a good signal to noise ratio has to be larger than 100 nV, and this is ten times higher than the amplifier's internal noise.

The IA self-noise can be reduced many times by decreasing the circuit's temperature whenever such extremely high sensitivity is desired. This can be achieved by cooling the device through keeping the circuits inside a liquid nitrogen container. This way highly reduces the self-noise of the circuits by means of reducing the thermal, Johnson, and flicker noises.

If the sensitivity of about 1 PPM is required, and the IA has 100 nV minimum input voltage, therefore the bi-directional coupler should produce 0 Volt DC value for the VF.
VF=0.1V=100 nV×1,000,000

The 0.1V forward voltage embodies the 50-ohm (Ω) impedance of a matched load, consequently the 100 nV variations (wavering) in the VF voltage can be considered argumentatively to represent a 50 micro-ohm (μΩ) impedance variations in the same load.
50 μΩ=50 Ω/(0.1V/100 nV)=50 Ω/1E6

This general way of assumption is not so accurate; since there is no such accurate tool available at the present days that could be relied on to measure such very low impedance variations.

The practical observations demonstrated that the actual impedance sensitivity for a matched load is much better than what have been estimated above, because working at a good degree of impedance match of 1 to 1.5 VSWR (voltage standing wave ratio), where the curve for the VF and VR becomes exponentially sharp as depicted in FIG. 9, the slightest changes in the load impedance will lead to the highest changes for the VF and the VR values. The sensitivity decreases by increasing the degree of impedance match and vice versa.

Moreover increasing the DC-Voltage value of VF12 increases the final system sensitivity. Due to the coupling coefficient limitation, the increase of EMW power being transmitted has been demonstrated to be the proper solution that increases the DC-Voltage of VF.

For example if the IA has 100 nV minimum input voltage, and if the DC value for the VF equals 10V, therefore the resulted sensitivity will be:
Sensitivity=10V/100 nV=10 PPB.

Reducing the IA self-noise and enhancing the purity of the EMW can highly improves the final system sensitivity.

Actually in some cases it is not recommended to increase the transmitted power, for example in portable applications the power supply consumption is a very important factor, that's why the output power should be reduced.

Also small insects cannot tolerate high RF powers while monitoring the their bioactivities. Also for a safety reason, according the regulations of the Federal Communications Commission/USA, the maximum permissible uncontrolled power exposure at 30 MHz for a period of 30 minutes, should not exceed 180 mW/cm².

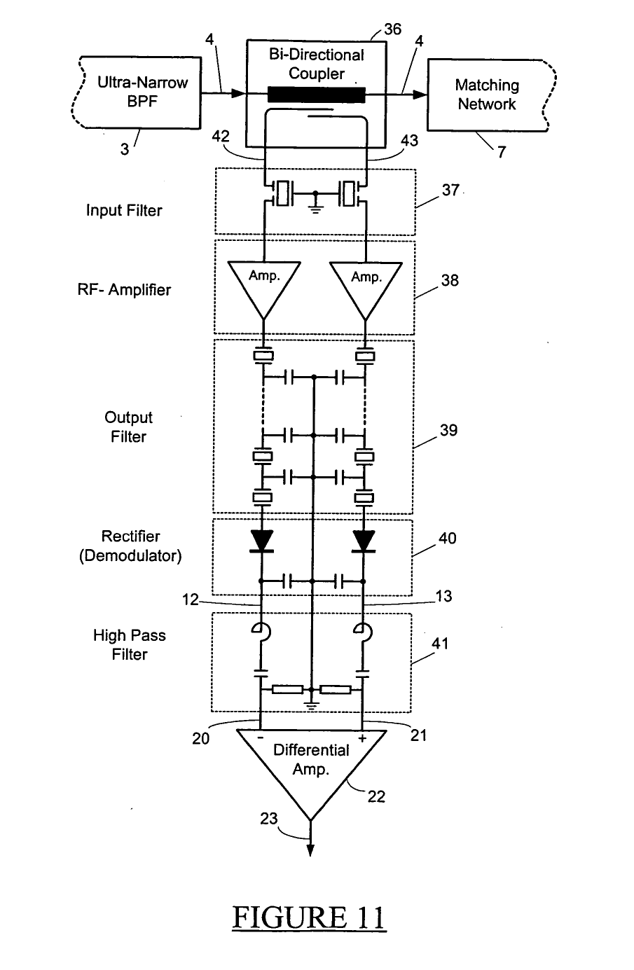

Therefore acquiring a high DC Voltage value for the VF from a low transmitted power is possible by using a bi-directional coupler 36 that has un-rectified outputs as depicted in FIG. 11, this means that the forward 42 and reflected 43 powers have to remain in there HF format without any demodulation. This enables the use of an ultra-narrow band (selective) RF amplifier 38. While these tiny powers remain in the HF format, the amplifying is possible without the risk of adding an extra noise generated by the amplifier's circuit 38 to the amplified signal 42, 43. Narrowing the bandwidth of the amplifier highly reduces the amplifier self-noise, and therefore enhances the amplified signal purity.

In low-power applications, the signal being tracked is lesser than the amplifier self-noise, therefore by using an ultra-low noise and selective RF amplifier 38 along with many ultra-narrow BPF 37, 39, (CLF, LC, and ceramic filter), the purity of the amplified signal remains as the un-amplified one.

Rectifying (demodulating) the amplified HF powers can be achieved by using dual matched Schottky type diodes along with fining capacitors 40. Using P-type zero bias Schottky detector diodes is necessary for achieving a high rectifying linearity in a wide range of input voltages, and because of their own low-flicker noise.

It has been noticed that the system is susceptible to rough vibration artifacts, which therefore affects the mechanical stability of the HF oscillator circuit, this is because the center frequency of crystal oscillator 1 is very susceptible to mechanical vibrations. Moreover the matching network 7 consisting of frequency dependent components (inductors and capacitors), so any changes in the oscillator frequency, leads to instability (deviation) in the resultant impedance match. Therefore the weight of the oscillator circuit should be lightweight as much as possible, it has to be surrounded and fastened inside the device by placing it in sponge compartment that establishes a vibration absorber.

In order to prepare the system to be implemented in any application, a few tunings and modifications are required. In general the essential preparations are based on choosing the proper transmitting antenna 48, which is used to introduce the EMW into the region of the body being monitored 11. Likewise it is necessary to define the preferred sensitivity, as well as adjusting the frequency bandwidth to cover the expected bioactivities being monitored.

The final sensitivity can be easily tuned by adjusting the gain of the output amplifier 27, and the frequency bandwidth can be tuned by adjusting the components of the active filter 26, or by modifying the parameters of the DSP software.

However the shape of Electrodes 10 generally determines the type of the intended application. Each application requires different electrodes 10 with different size, shape, and insulation thickness.

Monitoring the tiny superficial blood flow fluctuations within the concealed capillary vessels requires more attention. The capillary blood flow within the superficial vessels (skin) at rest, has estimated to be about 1 micro-liter per second (μL/S) for each square centimeter, also the actual fluctuation in the capillary blood flow doesn't exceed 10% of the total volume flow. Consequently the blood fluctuates by about 0.1 μL/S.

Moreover the hemodynamic cardiography monitors the instant capillary blood flow within the bandwidth of 0.1 to 100 Hz, therefore the upper frequency limit (100 Hz), which represents the fast blood flow variations, outlines the 1 nano-liter (peak to peak) variations in the blood volume for each 10 milli-second (nL/10 mS).

The transmitting antenna (probe) 48 which is intended to monitor the superficial bioactivity such as the capillary blood flow within the skin, comprises of dual symmetrical electrodes 10 (FIG. 8) made from thin sheet of metal, that have relatively a similar length (L) and width (W) of each electrode of about 2 to 5 mm, and a distance (D) between both electrodes of a few milli-meters. The insulation layer 29 can be made from any thin plastic or rubber sheet of less than 1 mm thickness, means to achieve proper electrical isolation.

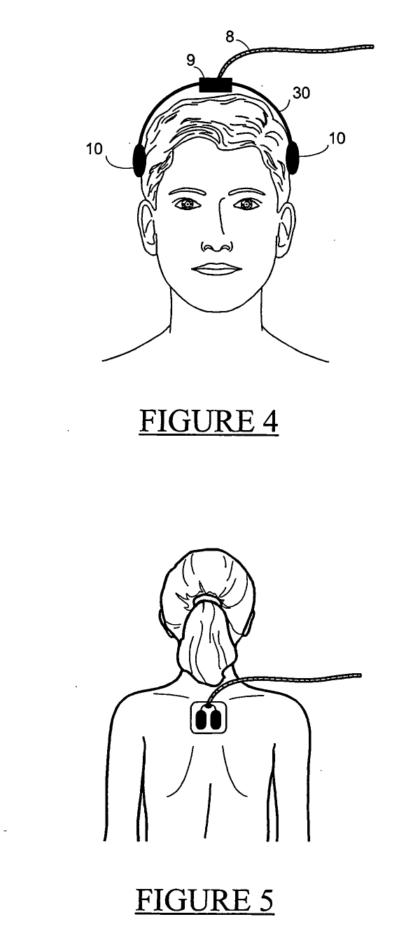

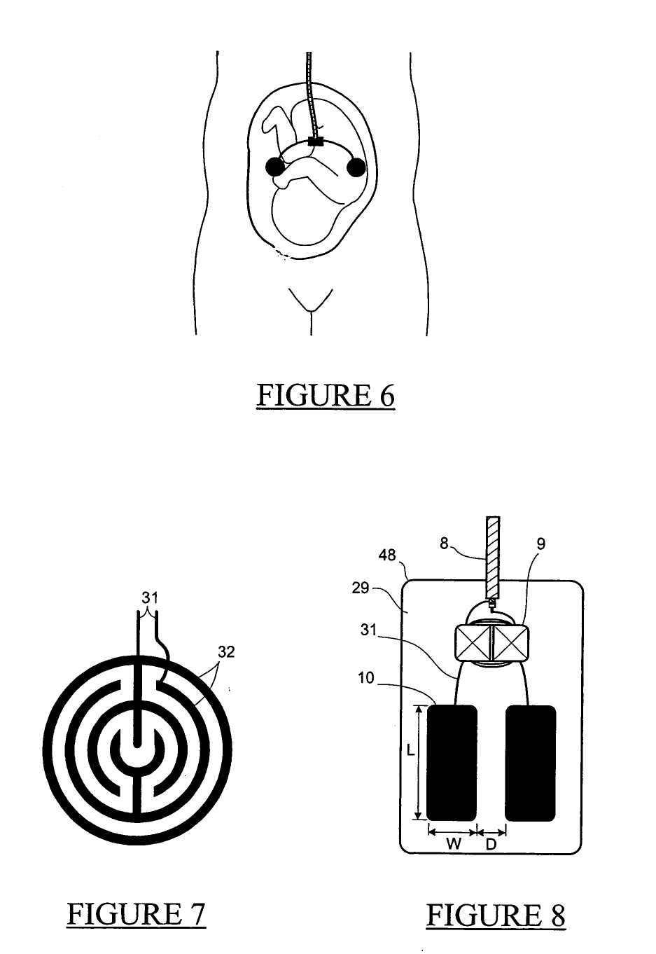

Monitoring a more deep bioactivity (brain, CNS, and fatal, FIGS. 4, 5, and 6) requires larger size electrodes 10 in the centimeter range, likewise an extended distance between the electrodes should be considered too, also a thicker insulation should be prepared.

The relationship between the bioactivity depth, electrode size, distance between electrodes, and the insulation thickness is a direct-proportional relationship. The purpose of using thick insulation is to reduce the sensitivity for the superficial bioactivity, and to increases the threshold sensation for a deeper bioactivity.

From the EMW propagation theory as depicted in FIG. 3 it is a well-known fact that EMW becomes attenuated by being away from the transmitting antenna, as well the direction of propagation turns to the surrounding objects that has the lowest impedance, wherein the surrounding objects luckily will act as a waves director. Therefore the EMW mainly propagates toward the nearest region of the body being monitored. Enlarging the electrode size will enlarge the inspected area being monitored. Likewise the distance enlargement between the electrodes enlarges the radius of the electromagnetic field being created.

The purpose of enlarging the insulator thickness is to keep electrodes away from the inspected region, to insure a deeper delivery of the EMW inside the body, and to reduce the high sensitivity (proximity effect) that occurs by positioning the electrodes very close to the body.

Moreover keeping electrodes away from that region which doesn't contain a large moving activity such as the skull, achieves another way of sensation to a deeper bioactivities that's directly affected by the impedance property of the cells, The cells (tissue) impedance varies from about 10 kΩ/cm at rest, to about 1 kΩ/cm at action. Therefore thickening the insulator 29 reduces the sensitivity to the tiny superficial bioactivities, and this is very important factor for monitoring the brain bioactivity without any significant interference with the natural blood flow within the skull. The same arrangement can be done to monitor the CNS communications within the upper side of the spinal cord (Medulla Oblongata).

Placing the region being monitored between a two opposite transmitting electrodes as depicted in FIG. 3 insures the highest possible sensitivity, but such positioning reduces the impedance pre-matched flexibility from being always ready for use in a different patient circumstances, this means that the distance between the opposite electrodes is defined by the thickness of the inspected region which varies between peoples, and therefore this varies the resultant load impedance, consequently any achieved impedance match cannot be valid for different positioning of electrodes. Therefore this will force to employ an auto-tuning matching network, instead of permanent matching network 7, which is applicable for many positioning circumstances. By employing the adjacent type transmitting electrodes as depicted in FIG. 8, it is not required to retune the matching network 7 every time.

When the system has to be used out of clinic, by transmitting the patient cardiograms to a receiving unit that located in a hospital's emergency, also as in out-patient monitoring system which is used in the ambulance or the rescue helicopter, and the tele-patient monitoring (Bluetooth® cardiography), or the soldier of future, were it is necessary to keep watching the soldier health remotely within the battlefront. In such applications, which are running in shaking conditions that release many vibration artifacts, extra measures to maintain the monitoring stability are required.

The effect of vibration artifacts can be highly reduced by eliminating the output transmitting cable 8, by means of connecting directly the sensing probe (antenna electrodes) 48 to the matching network 7, this is to eliminate the vibration effect, which occurred due to the swinging in the transmitting cable 8. Furthermore minimizing the device or at least the HF blocks (1-10) to a size that can be fit in the belt or the bracelet, which is fastened around the region of the body being monitored.

If it is needed to achieve multi-channel monitoring system as depicted in FIG. 10, as for the multidirectional monitoring of the brain bioactivities, in a similar way of the traditional EEG. The produced power of EMW can be divided to many symmetrical ports that each port continues independently to all of the following stages. Individual CLF 34 is sufficient for each port, because the 10 mW of EMW power is enough and sufficient for driving each port.

A dual-channel monitoring system is effective for subtracting the undesired signals, for example if the blood's flow affects the signal being obtained while monitoring the CNS bioactivities, an additional sensor can be used to monitor only the blood flow in that region of the body which doesn't contain any other activities, and then the resulted blood flow signal can be subtracted from the first signal being obtained by the CNS sensor. By this way, monitoring specific activity is possible even if it is founded in a region that contains undesired artifacts. Likewise it can help in monitoring the fetal activities without being influenced by the mother bioactivities.

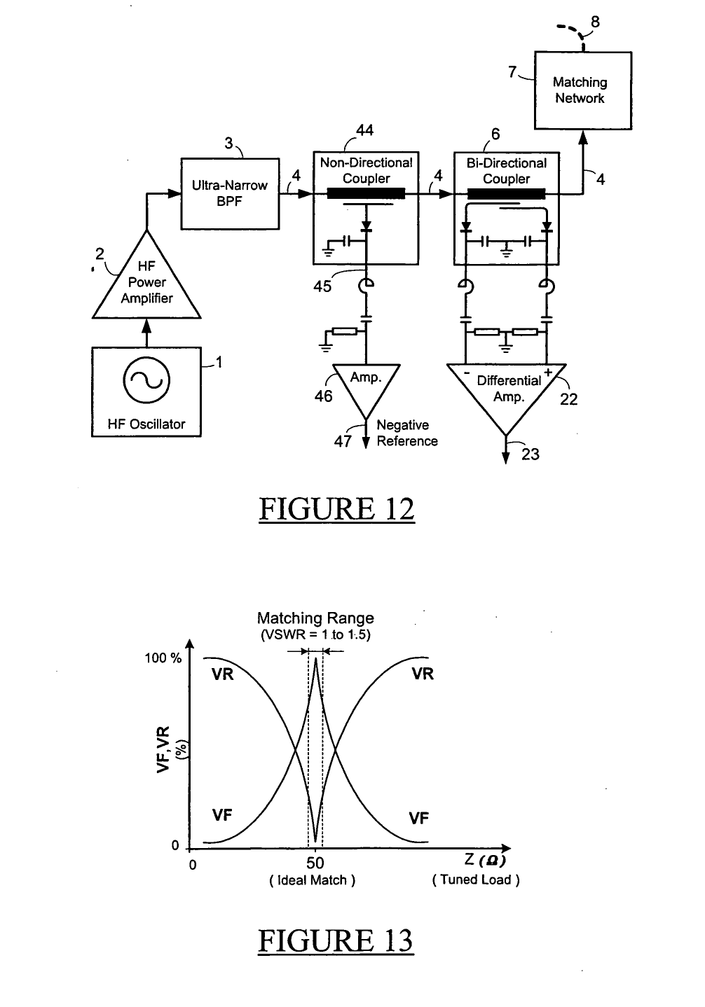

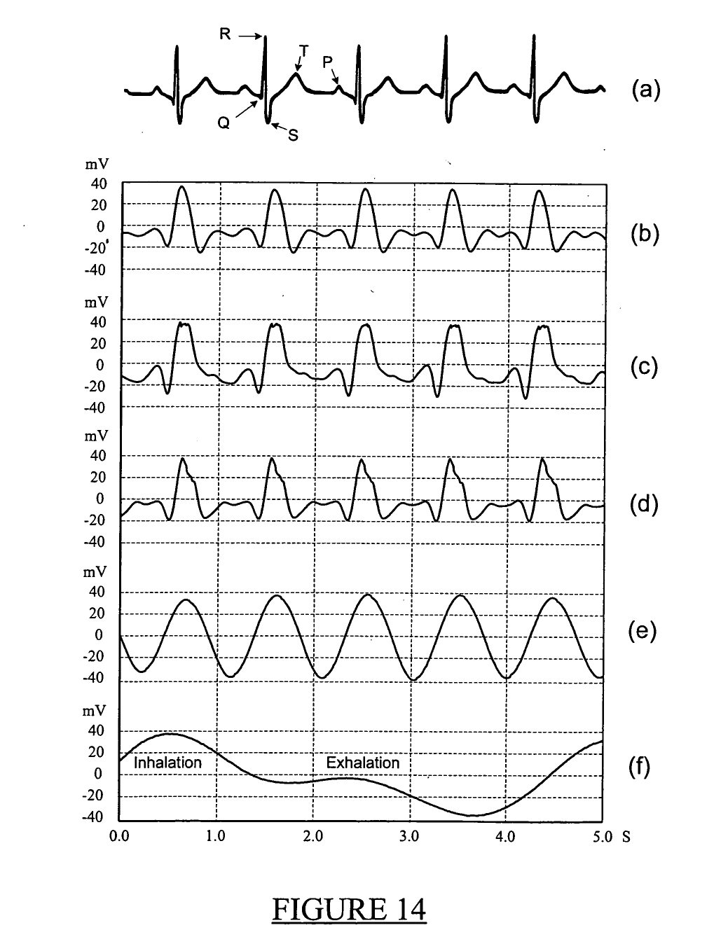

Reducing the effect of internal noise and instability on the final sensitivity can be achieved by adding a non-directional coupler 44 within the same transmission line 4 as depicted in FIG. 12. The output signal 45 of the non-directional coupler after being rectified is used to estimate the instability of the produced EMW. The output signal 45 of the non-directional coupler 44 has no phase characteristics, and therefore it reflects only the amplitude instability of the produced EMW. By extracting the variable components through the use of DC blocking capacitor, the amplified signal 47 will contain the necessary data about the HF instability, and so it can be used as a negative reference in the final signal processing stage.

The very high sensitivity is required by a few limited applications, such as for monitoring the bioactivities of the very small insects like the ants. For a two-centimeter cockroach, a sensitivity of few PPM is sufficient to acquire good satisfying results.

At this time it is impossible to apply this technology for monitoring the bioactivity of an individual cell. This technology is still nascent, and it is intended now for monitoring the bioactivities for a large amount of cells that are combined in the tissue. In the near future and by the presented technology, the sensitivity could reach the capability of monitoring even the plant leaf bioactivities.

1. Apparatus for non-invasively (non-contact) monitoring the cellular bioactivities in a body, said apparatus comprising:
High frequency (HF) power oscillator means for producing stable sinusoidal HF electromagnetic (EM) energy; and
Balanced type antenna means for introducing the EM energy being produced by the said oscillator into the region of the body being monitored; and
Matching network means for matching the output impedance of the said oscillator means with the characteristic impedance of the said region of the body being monitored; and
Ultra narrow band pass filter (BPF) means for reducing the internal random noises which contaminates the said produced EM energy; and
Bi-directional coupler connected in series within transmission line means for instant sampling the internal forward and reflected power values that only occurred inside the said transmission line due to the transmission lines match and mismatch phenomena; and
Dual high pass filters (HPF) means for filtering the said forward and reflected power values means for passing only the variable voltages which contains the useful indications about the Impedance match and mismatch variations between the said region of the body being monitored and the said power oscillator; and
Differential amplifier means for differentially combining the said extracted variable voltages means for producing an output voltage that contains an indications about the cells bioactivity being monitored; and
Analogue divider means for dividing the said resulted differential voltage to at least one output that maintaining the same characteristics as the undivided input; and
Active filter(s) means for separating specific frequencies bandwidth by band(s) pass and/or band(s) reject filtering the said divided voltage(s) means for extracting different kinds of cells bioactivities.
Output amplifier(s) means for amplifying the said actively filtered voltage to the needed level sufficient to drive the following analytical/indicator circuits

2. A method for non-invasively (non-contact) monitoring cellular bioactivities in a body by means of monitoring impedance variations that occur due to the movements of ionized particles across the cells membrane and/or by means of monitoring the impedance variations that occur due to the cellular movement or flow within the body, and/or by means of monitoring changes in the cellular impedance which occur due to differential concentration of the ionized particles on both sides of the cellular membrane, the method comprising:
Directing EMW being produced by HF power oscillator to the region of the body being monitored by Balanced type antenna; and
Matching the output impedance of the said HF power oscillator and the characteristic impedance of the said region of the body being monitored; and
Ultra narrow band pass filtering means for purifying the produced EMW; and
Instant sampling, via a Bi-directional coupler, of the forward and reflected power values which occur inside transmission line due to the transmission lines match and mismatch phenomena; and
High pass filtering the said forward and reflected power values means for passing only the variable voltages that contain indications concerning the Impedance match and mismatch variations between the said region of the body being monitored with the said oscillator; and
Differentially combining the said variable voltages means to produce differential output voltage that contain indications pertaining to the cellular bioactivity being monitored; and
Separating specific frequency bandwidths by band(s) pass and/or band(s) reject filtering of the said differential voltage means to extract data relating to specific types of cellular bioactivities

3. A device comprising:
HF oscillator for producing EM-waves for non-invasively (non-contact) monitoring the cells bioactivity, the said oscillator possesses a power range from 1 to 100 milli-watt, and a frequency range from 1 to 300 MHz; and
Balanced type antenna comprised of dual insulated parallel or opposite or overlapped metal sheets or wires, the said antenna being adapted for any location on the region of the body containing the collection of cells to be monitored; and
An impedance matching network means for matching the output impedance of the HF oscillator with the characteristic impedance of the region of the body being monitored; and
Means for extracting variable voltages from the forward and reflected transmission line voltages to produce voltages which provide indication data about the impedance variations of the region of the body being monitored; and
Means for differentially combining the said extracted variable voltages to obtain a differential signal which is indicative of cellular bioactivities within the region of the body being monitored; and
Means for separating the said differential signal via frequency bandwidth means to ascertain specific cells bioactivity; and
Means for amplifying the said separated signal to a level that can drive the output analytical or indicator circuits.

4. A device as claimed in claim 3, wherein said device is adapted for non-contact monitoring the body hemodynamics, means for monitoring the vital activities of the heart and/or the lung and/or the vascular system.

5. A device as claimed in claim 3, wherein said device is adapted for non-contact monitoring the brain bioactivities.

6. A device as claimed in claim 3, wherein said device is adapted for non-contact monitoring the bioactivities of the central and/or peripheral nervous system.

7. A device as claimed in claim 3, wherein said device is adapted for non-contact monitoring vital sign activities of fetal organs.

8. A device as claimed in claim 3, wherein said device is adapted for non-contact monitoring insect bioactivities.
